(19) United States
(12) Patent Application Publication
(10) Pub. No.: US 2009/0035373 A1
(43) Pub. Date: Feb. 5, 2009

(54) PHARMACEUTICAL COMPOSITION FOR CONTRACEPTION AND BLOOD PRESSURE REGULATION, KIT FOR CONTRACEPTION CONTAINING SAME AND METHOD OF PRODUCTION OF SAME

(76) Inventors: Katrin Mittmann, Jena (DE); Thomas Zimmermann, Jena (DE)

Correspondence Address: Striker, Striker & Stenby, 103 East Neck Road, Huntington, NY, 11743, US

(21) Appl. No.: 12/135,470

(22) Filed: Jun. 9, 2008

Related U.S. Application Data
(60) Provisional application No. 60/952,940, filed on Jul. 31, 2007.

(51) Int. Cl. A61K 9/24; A61K 31/565; A61P 7/00; A61P 15/18

(52) U.S. Cl. 424/472; 514/170

(57) ABSTRACT

The pharmaceutical composition for contraception and regulating blood pressure includes daily dosage units, which contain 2.0 mg of dienogest and 0.030 mg of ethinyl estradiol, 2.0 mg of dienogest and 0.020 mg of ethinyl estradiol, or 1.5 mg of dienogest and 0.015 mg of ethinyl estradiol, together with at least one excipient and/or carrier. Each daily dosage unit may be a coated tablet for oral administration consisting of a tablet core and a coating around the core. The coating contains all of the ethinyl estradiol and a part of the dienogest, which are released in a non-retarded fashion. The tablet core contains another part of the dienogest that is released in a retarded fashion. The risk of folate deficiency-induced congenital malformations in the event of pregnancy may be reduced by including (6S)-5-methyltetrahydrofolate in each daily dosage unit. A kit for a 28-day cycle is also disclosed.

### CROSS-REFERENCE

The invention described and claimed herein below is also described in U.S. Provisional Patent Application, Ser. No. 60/952,940, which was filed on Jul. 31, 2007 in the U.S. Patent Office. The foregoing U.S. Provisional Patent Application provides a basis for a claim of priority for the invention described and claimed herein below under 35 U.S.C. 120.

### BACKGROUND OF THE INVENTION

1. The Field of the Invention

The present invention relates to pharmaceutical preparations for contraception and for blood pressure regulation, to methods of producing them and to kits for contraception containing them.

2. Description of the Related Art

The significance of cardiovascular disease as a health problem is under-estimated in many cases. For example, 35% of potential patients in the USA consider breast cancer to be their highest health risk and only 7% believe that it is cardiovascular disease.

Hypertension is one of the decisive risk factors for cardiovascular disease. Hypotension, however, should not be disregarded either, because it can have an appreciable effect on the quality of life of the affected individual via its symptoms, such as fatigue, loss of drive, weakness, dizziness, and a tendency to loose consciousness.

Excessively low blood pressure can lead to insufficient blood supply and thus insufficient oxygen supply to the heart, brain and other organs. For pregnant women, low blood pressure is a risk factor, because a causal connection exists between reduced blood pressure, insufficient blood circulation in the uterus, development disturbances and perinatal complications.

Naturally, both hypotension and hypertension should be treated with appropriate means primarily by a cardiologist. However the connection between hypertonia/hypotonia and contraception, hormone replacement, and the treatments of gynecological diseases should be considered more intensively by gynecologists.

A. O. MUECK, et al, in “Hormone Therapy and Hypertonia”, J. für Hypertonia, 10 (1), (2006), pp. 14-21 describes the evaluation of blood pressure in the current classification of the American Heart Association as follows:

| | Systolic, mm Hg | | Diastolic, mm Hg |
|---|---|---|---|
| Optimal | <120 | and | <80 |
| Normal | <130 | and | <85 |
| Borderline normal | 130-139 | or | 85-89 |
| Hypertonia | | | |
| Stage 1 (mild) | 140-159 | or | 90-99 |
| Substage: “borderline” | 140-149 | or | 90-94 |
| Stage 2 (moderate) | 160-179 | or | 100-109 |
| Stage 3 (severe) | ≧180 | or | ≧110 |
| Systolic, isolated | ≧140 | and | <90 |

The foregoing blood pressure values correspond to those given in European Society of Hypertension, 2003; Categorized Blood Pressure Values:

| | Systolic | Diastolic |
|---|---|---|
| Category | (mm Hg) | (mm Hg) |
| Optimum values | <120 | <80 |
| Normal values | 120-129 | 80-84 |
| High normal values | 130-139 | 85-89 |
| Stage 1 hypertonia (mild) | 140-159 | 90-99 |
| Stage 2 hypertonia (moderate) | 160-170 | 100-109 |
| Stage 3 hypertonia (severe) | ≧180 | ≧110 |
| Isolated systolic hypertonia | ≧140 | <90 |

For women, hypotonia (low blood pressure)—at first only a measured parameter and not a disorder—is defined by the World Health Organization WHO as a blood pressure of less than 100/60 mg.

A connection is known between the administration of oral contraceptives and both hypertonia and hypotonia.

Oral contraceptives are known to raise the blood pressure. For example, R. G. KETELHUT, in “Long-term Discontinuation of Oral Contraceptives”, J. für Hypertonie, 4(2), (2000), pp. 18-21, showed that blood pressure dropped after long-term discontinuation of administration of oral contraceptives. H. D. TAUBERT, et al, in “Kontrazeption mit Hormonen [Contraception with Hormones]”, 2^{nd} re-worked and expanded ed., publ. by Georg Thieme Verlag, Stuttgart, N.Y., (1995), pp. 273-275, showed that administration of contraceptives raises blood pressure and that estrogens, such as ethinyl estradiol (EE), are responsible for the rise in the blood pressure. F. LEIDENBERGER, et al (editors), “Klinische Endokrinologie für Frauenarzte [Clinical Endocrinology for Gynecologists]”, 3^{rd} complete and expanded ed., pubi. by Springer Medizin Verlag, Heidelberg, (2005), p. 192, found that the increase in blood pressure that takes place due to administration of oral contraceptives is brought about, for example, by the action of ethinyl estradiol (EE) on the renin-angiotensin system, but not by estradiol. Oral contraceptives increase the blood pressure, especially after long-term administration, and ethinyl estradiol (EE) was assumed until now to cause the increase of the blood pressure, according to A. O. MUECK, et al, ibid.

Other investigators, for example M. GRUBER, in “Drospirenone—The Gestagen Makes the Difference”, in J. für Fertilitat und Reproduktion, 13(3), (2006), p. 19-20 and N. OELKERS, “Drospirenone: A New Gestagen” in Geburtshilfe und Frauenkunde 61, (2001), pp. 756-851, have shown that administration of estrogens, especially ethinyl estradiol (EE), raises the blood pressure.

Drospirenone (DRSP)—a spironolactone of the aldosterone antagonist type—can be used as an oral contraceptive according to DE 30 22 337, published on Jan. 7, 1982. It was found that DRSP has a progesterone-like action mechanism. Earlier work described in DE 26 52 761 A, published May 18, 1978, showed that DRSP has diuretic properties of the aldosterone antagonist type. U.S. Pat. No. 6,787,531 of Aug. 31, 2000 described work that showed that DRSP has diuretic properties and counteracts the water-retention properties of ethinyl estradiol (EE).

Drospirenone (DRSP) has been shown to forestall the increase in blood pressure caused by administration of EE on account of its aldosterone-antagonistic effect by N. OELKERS, ibid. Also M. GRUBER, et al, ibid, showed that DRSP regulates blood pressure. However drospirenone (DRSP) was shown to cause no change in the blood pressure in normo-tensive and hypertensive post-menopausal women, according to A. O. MUECK, et al, ibid.

When the combination of DRSP and E2 is administered for hormone replacement, the sodium retention and water retention and consequent heavy swollen legs are reduced as a result of the aidosterone receptor antagonism of DRSP, according to N. KALUS, “ANGELIQ®—First Hormone Replacement Therapy with a Gestagen Showing an Aldosterone-antagonistic Effect”, in J. for Menopause, 3, (2004), pp. 20-21. The combination comprised 1 mg of E2 and 2 mg of DRSP. The aldosterone-antagonistic mechanism of the gestagen counteracted the normal sodium and water retention action of the estrogen. Similar results are described in U.S. Pat. No. 6,787,531 of Aug. 31, 2000 and E. BOSCHITCH, “Hormone Replacement—Drospirenone” in Speculum, Z. für Gynäkologie und Geburtshilfe, 22(4), (2204), pp. 35-45.

Administration of a combination of DRSP and E2 (natural estrogen—estradiol) reduces the systolic and diastolic blood pressure, because of the aldosterone receptor antagonism of DRSP according to W. BRAENDLE, “New Oral Contraceptive Containing an Antimineralocorticoid and Antiandrogenic Gestagen” in Geburtshilfe und Frauenkunde 61, (2001), pp. 101-105. Activation of the rennin angiotensin-aldosterone system in the kidney caused the antimineralo-corticoid effect of DRSP according to this prior art reference. The presence of ethinyl estradiol (EE) did not affect the action of the combination. These results were confirmed and extended by E. BOSCHITCH, ibid, who found that aldosterone receptor antagonists, such as DRSP, have an antihypertensive effect and a diuretic effect, even in combination with estradiol.

Administration of a combination of DRSP and E2 (natural estrogen—estradiol) in combination with a blood pressure inhibitor was found to reduce blood pressure or enhance the action of the inhibitor in post-menopausal women according to E. A PRESTON, “Effects of Blood Pressure Reduction” in Climacteria and E. BOSCHITCH, ibid. U.S. Published Patent Application 2004/034001 and WO 2003/090755 (EP 1 499 323) disclose that DRSP/estrogen (EE, E2) alone, or together with blood pressure inhibitors, reduces blood pressure and can be used in HRT.

U.S. Published Patent Application 2004/0087563 and WO 2004/041289 (EP 1 558 265) disclose that gestagens with antimineralocorticoid activities, such as DRSP, in combination with an estrogen and in combination with gestagens with non-antimineralocorticoid activities (dienogest—DNG), are used in the treatment of aldosterone-induced disorders, including blood pressure.

### SUMMARY OF THE INVENTION

It is an object of the present invention to provide a single pharmaceutical preparation that has both contraceptive action and blood pressure-regulating action.

It is a further object of the present invention to provide a method of making the single pharmaceutical preparation that has contraceptive action and blood pressure-regulating action.

It is an additional object of the present invention to provide a kit for contraception that comprises daily dosages of the pharmaceutical preparation according to the present invention.

According to the present invention one or more of the above-described objects of the present invention are attained by a pharmaceutical composition for blood pressure regulation and contraception comprising a daily dose unit, which contains:

2.0 mg of 17α-cyanomethyl-17-β-hydroxyestra-4, 9-dien-3-one (dienogest), and 0.030 mg of 17α-ethinyl estradiol (ethinyl estradiol), or

2.0 mg of dienogest and 0.020 mg of ethinyl estradiol, or

1.5 mg of dienogest and 0.015 mg of ethinyl estradiol;

and one or more pharmaceutically acceptable excipients/carriers. Preferred embodiments of this pharmaceutical composition comprise a coated tablet consisting of a tablet core containing a part of the total amount of dienogest, which is to be released in a retarded fashion, and a coating around the tablet core, which contains a non-retarded (rapidly) released part of the total amount of dienogest and a non-retarded (rapidly) released total amount of the ethinyl estradiol. At least 10%, and preferably 30%, of the dienogest present is dissolved out of the tablet core in a retarded fashion after more than 30 minutes, as determined by a dissolution test with water at 37° C. as dissolution medium and a stirring rate of 50 rpm/min.

In further preferred embodiments the daily dose of the pharmaceutical composition contains (6S)-5-methyltetrahydrofolate, preferably from 0.4 to 1 mg, especially 451 μg, of the crystalline or microencapsulated calcium salt of (6S)-5-methyltetrahydrofolic acid in various suitable crystal forms.

According to the present invention one or more of the above-described objects of the present invention are attained by a kit containing

at least 21 daily dose units of the aforesaid pharmaceutical composition, each of which contains one of the aforesaid combinations of dienogest and ethinyl estradiol, and

at the most 7 daily dose units containing no active agent or containing a placebo.

In preferred embodiments of the aforesaid kit the number of daily dose units containing the combination of dienogest and ethinyl estradiol amounts to 21, 22, 23, 24, or 25 and the number of daily dose units containing no active agent is 7, 6, 5, 4, or 3 so that the kit contains a total number of 28 daily dose units corresponding to the number of days in a 28-day cycle.

According to the present invention one or more of the above-described objects of the present invention are attained by a kit containing

at least n×21 daily dose units of the aforesaid pharmaceutical composition, each of which contains one of the aforesaid combinations of dienogest and ethinyl estradiol, in which n is 2, 3, 4, 5, 6, 7, 8, 9, 10, 11, 12, 13, 14, 15, 16, or 17, and

at the most 7 daily dose units containing no active agent or containing a placebo.

Preferably the number of daily dose units containing no active agent or containing a placebo is 3, 4, 5, 6, or 7.

In a preferred embodiment of the kit the total number of daily dose units containing the combination of dienogest and ethinyl estradiol amounts to 84 and the number of daily dose units containing no active agent or containing a placebo is 7 so that the total number of cycle days per year is 4×(n×21 plus 7), n being equal to 4.

Preferred embodiments of the kit have daily dose units containing 451 μg of the crystalline or microencapsulated calcium salt of (6S)-5-methyltetrahydrofolic acid.

According to the present invention one or more of the above-described objects of the present invention are attained by a process of preparing a pharmaceutical composition for blood pressure regulation, for oral contraception and simultaneous blood pressure regulation, or for reducing the risk of congenital disorders in unborn children (fetuses) and the risk of perinatal complications in the event of pregnancy, which process comprises including

a combination of 2.0 mg of dienogest and 0.030 mg of ethinyl estradiol, or

a combination of 2.0 mg of dienogest and 0.020 mg of ethinyl estradiol, or

a combination of 1.5 mg of dienogest and 0.015 mg of ethinyl estradiol, in the pharmaceutical composition.

In a preferred embodiment of the aforesaid process a single phase pharmaceutical composition for oral administration is made by the process.

According to the present invention one or more of the above-described objects of the present invention are attained by a process of preparing a pharmaceutical composition for oral contraception and simultaneous blood pressure regulation and for reducing the risk of folate deficiency-induced congenital malformations in the event of pregnancy, which process comprises including

a combination of 2.0 mg of dienogest, 0.030 mg of ethinyl estradiol, and (6S)-5-methyltetrahydrofoiate, or

a combination of 2.0 mg of dienogest, 0.020 mg of ethinyl estradiol, and (6S)-5-methyltetrahydrofolate, or

a combination of 1.5 mg of dienogest and 0.015 mg of ethinyl estradiol, and (6S)-5-methyltetrahydrofolate, in the pharmaceutical composition.

Preferred embodiments of the process of making the aforesaid pharmaceutical composition include 451 μg of the crystalline or microencapsulated calcium salt of (6S)-5-methyltetrahydrofolic acid in the pharmaceutical composition.

It must be kept in mind that the above-described inventive concept refers mainly to the “high normal values” to “mild hypertonia-stage 1 and “borderline” groups of individuals according to the blood pressure categories summarized in the section described prior art, because for safety reasons the test subjects of the moderate and severe hypertonia groups “stages 2 and 3” are not included into clinical studies that test the effectiveness of contraceptives.

### EXAMPLES

The following examples illustrate the above-described invention in more detail, but the details in these examples should not be considered as limiting the claims appended herein below.

### Example 1

Valette is a conventional sugar-coated tablet for oral contraception containing 0.030 mg of ethinyl estradiol and 2.0 g of dienogest in a tablet core covered by a sugar-containing coating.

### Example 2

2 mg of dienogest and 0.02 mg of ethinyl estradiol, with 1 mg of dienogest, which is released in a retarded fashion, and 1 mg of dienogest and 0.02 mg of ethinyl estradiol, which are rapidly released.

Description:

The example is a coated tablet with a matrix core. The core of the coated tablet contains 1 mg of dienogest in a hydrophilic erosion matrix with METOLOSE® as the basic constituent. The matrix releases the active ingredient, dienogest, in a retarded manner. The core was coated with a rapidly dissolving coating containing 1.0 mg of dienogest and 0.02 mg of ethinyl estradiol. For protection against light, the coated tablet was covered with an additional, rapidly dissolving colored layer containing iron oxide pigments.

The following table I describes the composition of the tablet core and tablet coating of example 2.

TABLE I
COMPOSITION OF THE TABLET CORE AND
TABLET COATING OF EXAMPLE 2

| CORE | | | COATING FILM | | |
|---|---|---|---|---|---|
| | | | Film 1-containing | | |
| Granulate 1: | | | the active ingredient: | | |
| Dienogest | 1.000 | mg | Dienogest | 1.000 | mg |
| METOLOSE ® 90 | 7.500 | mg | Ethinyl estradiol | 0.020 | mg |
| SH-4000 | | | | | |
| Lactose monohydrate | 21.000 | mg | METHOCEL ® 5 | 2.250 | mg |
| Corn starch | 14.000 | mg | Talc | 0.0450 | mg |
| Povidone K25 | 1.500 | mg | Titanium dioxide | 0.280 | mg |
| (10% in ethanol) | | | | | |

| Granulate 2: | | | Film 2-Colored layer: | | |
|---|---|---|---|---|---|
| Lactose monohydrate | 54.000 | mg | METHOCEL ® 5 | 3.375 | mg |
| Corn starch | 27.100 | mg | Talc | 0.675 | mg |
| Maltodextrin | 6.900 | mg | Titanium dioxide | 1.875 | mg |
| (25% in water) | | | Iron oxide, red | 0.075 | mg |

| | | |
|---|---|---|
| Outer phase: | | |
| Carboxymethylstarch | 1.500 | mg |
| sodium | | |
| Magnesium stearate | 1.500 | mg |

### Example 3

### Description:

The coated tablet, which contains a total dose of 1.5 mg of dienogest and 0.015 mg of ethinyl estradiol, consists of a retarded matrix core and a rapidly dissolving coating, as well as a colored layer.

The following table II describes the composition of the tablet core and tablet coating of example 3.

TABLE II
COMPOSITION OF THE TABLET CORE AND
TABLET COATING OF EXAMPLE 3

| | | |
|---|---|---|
| VARIANT | 8.5 | |
| CORE | 104 | mg |
| Dienogest | 0.675 | mg |
| METOLOSE ® 90SH-4000 | 9.000 | mg |
| Lactose monohydrate | 42.925 | mg |
| Corn starch | 15.000 | mg |
| Povidone K25 (19% in water) | | |
| Maltodextrin (25% in water) | 6.000 | mg |
| TABLETTOSE ® | 8.500 | mg |
| AVICEL ® PH 102 | 7.000 | mg |
| Magnesium stearate | 0.900 | mg |
| Barrier layer: | | |
| EUDRAGIT ® RL 30 D (as coating drier) | | |
| Macrogol 6000 | | |
| Talc | | |
| Active ingredient-containing coating: | | |
| Dienogest | 0.825 | mg |
| Ethinyl estradiol | 0.015 | mg |
| METHOCEL ® 5 | 4.060 | mg |
| Talc | 0.836 | mg |
| Titanium dioxide | 0.264 | mg |
| Colored layer: | | |
| METHOCEL ® 5 | 1.500 | mg |
| PEG = Macrogol 6000 | 0.300 | mg |
| Talc | 0.300 | mg |
| Titanium dioxide | 0.850 | mg |
| Iron oxide, red | 0.050 | mg |

### Example 4

Tablets Having the Following Composition were Prepared:

CORE

| | | |
|---|---|---|
| Dienogest | 2.000 | mg or |
| | 1.500 | mg |
| Ethinyl estradiol | 0.015 | mg |
| METAFOLIN ® | 0.451 | mg |
| Lactose monohydrate | 28.720 | mg |
| Corn starch | 15.000 | mg |
| Maltodextrin | 3.750 | mg |
| Magnesium stearate | 0.500 | mg |

An ethinyl estradiol-betacyclodextrin complex can be used in place of ethinyl estradiol. In the event that ethinyl estradiol-beta-cyclodextrin complex (1:2) is used, a maximum or about tenfold amount is to be used.

The substances are mixed in an appropriate fashion and granulated. At the end of the granulation process, METAFOLIN® is applied, the composition is once again mixed, and is then tableted and optionally coated.

### Example 5

Tablets Having the Following Composition were Prepared:

CORE

| | | |
|---|---|---|
| Dienogest | 2.000 | mg |
| Ethinyl estradiol | 0.030 | mg |
| METAFOLIN ® | 0.451 | mg |
| Lactose monohydrate | 28.720 | mg |
| Corn starch | 15.000 | mg |
| Maltodextrin | 3.750 | mg |
| Magnesium stearate | 0.500 | mg |

An ethinyl estradiol-beta-cyclodextrin complex can be used in place of ethinyl estradiol. In the event that ethinyl estradiol-beta-cyclodextrin complex (1:2) is used, a maximum or about tenfold amount is to be used.

The substances are mixed in an appropriate fashion and granulated. At the end of the granulation process, METAFOLIN® is applied, the composition is once again mixed and is then tabletted and optionally coated.

While the invention has been illustrated and described as embodied in a pharmaceutical preparation for contraception and for blood pressure regulation, a method for producing it and a kit for contraception containing it, the invention is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A pharmaceutical composition for blood pressure regulation and contraception, said pharmaceutical composition comprising a daily dose unit, which contains:
2.0 mg of dienogest and 0.030 mg of ethinyl estradiol, or
2.0 mg of dienogest and 0.020 mg of ethinyl estradiol, or
1.5 mg of dienogest and 0.015 mg of ethinyl estradiol;
and one or more pharmaceutically acceptable excipients and/or carriers.

2. The pharmaceutical composition as defined in claim 1, comprising a coated tablet, wherein said coated tablet consists of a tablet core and a coating extending around the tablet core, said tablet core contains a part of an entire amount of the dienogest in the coated tablet, which is released in a retarded fashion, and said coating contains another part of the entire amount of the dienogest, which is released in a non-retarded fashion, and an entire amount of said ethinyl estradiol, which is released in a non-retarded fashion.

3. The pharmaceutical composition as defined in claim 2, wherein at least 10% of said dienogest is dissolved out of said tablet core in said retarded fashion after more than 30 minutes, as determined by a dissolution test with water at 37° C. as dissolution medium and with a stirring rate of 50 rpm/min.

4. The pharmaceutical composition as defined in claim 2, wherein at least 30% of said dienogest is dissolved out of said tablet core in said retarded fashion after more than 30 minutes, as determined by a dissolution test with water at 37° C. as dissolution medium and with a stirring rate of 50 rpm/min.

5. The pharmaceutical composition as defined in claim 1, wherein said daily dose unit contains (6S)-5-methyltetrahydrofolate.

6. The pharmaceutical composition as defined in claim 1, wherein said daily dose unit contains from 0.4 to 1 mg of a crystalline or microencapsulated calcium salt of (6S)-5-methyltetrahydrofolic acid in various suitable crystal forms.

7. The pharmaceutical composition as defined in claim 6, containing 451 μg of said calcium salt of said (6S)-5-methyltetrahydrofolic acid.

8. A kit containing
at least 21 daily dose units of a pharmaceutical composition, each of said daily dose units containing
2.0 mg of dienogest and 0.030 mg of ethinyl estradiol, or
2.0 mg of dienogest and 0.020 mg of ethinyl estradiol, or
1.5 mg of dienogest and 0.015 mg of ethinyl estradiol;
and one or more pharmaceutically acceptable excipients and/or carriers; and
at the most 7 daily dose units containing no active agent or containing a placebo.

9. The kit as defined in claim 8, containing 21, 22, 23, 24, or 25 of said daily dose units containing the combination of the dienogest and the ethinyl estradiol and 7, 6, 5, 4, or 3 of said daily dose units containing no active agent or containing said placebo, so that a total number of daily dose units amounts to 28, which corresponds to a 28-day cycle.

10. The kit as defined in claim 8, wherein each of said daily dose units contains 451 μg of a crystalline or a microencapsulated calcium salt of (6S)-5-methyltetrahydrofolic acid.

11. A kit containing
at least n×21 daily dose units of a pharmaceutical composition, n being equal to 2, 3, 4, 5, 6, 7, 8, 9, 10, 11, 12, 13, 14, 15, 16, or 17, each of said daily dose units containing
2.0 mg of dienogest and 0.030 mg of ethinyl estradiol, or
2.0 mg of dienogest and 0.020 mg of ethinyl estradiol, or
1.5 mg of dienogest and 0.015 mg of ethinyl estradiol;
and one or more pharmaceutically acceptable excipients and/or carriers; and
at the most 7 daily dose units containing no active agent or containing a placebo.

12. The kit as defined in claim 11, containing 3, 4, 5, 6, or 7 of said daily dose units containing no active agent or said placebo.

13. The kit as defined in claim 11, containing 84 of said daily dose units, said daily dose units comprising said dienogest and said ethinyl estradiol, and 7 of said daily dose units containing no active agent or containing said placebo, so that a total number of cycle days per year is 4×(n×21 plus 7), n being equal to 4.

14. The kit as defined in claim 11, wherein each of said daily dose units contains 451 μg of a crystalline or a microencapsulated calcium salt of (6S)-5-methyltetrahydrofolic acid.

15. A process of preparing a pharmaceutical composition for blood pressure regulation, for oral contraception and simultaneous blood pressure regulation, or for reducing the risk of congenital disorders in unborn children (fetuses) and the risk of perinatal complications in the event of pregnancy, said process comprising including a combination of 2.0 mg of dienogest and 0.030 mg of ethinyl estradiol, or a combination of 2.0 mg of dienogest and 0.020 mg of ethinyl estradiol, or a combination of 1.5 mg of dienogest and 0.015 mg of ethinyl estradiol, in the pharmaceutical composition.

16. The process as defined in claim 15, wherein the pharmaceutical composition is a single phase preparation and is designed for oral administration.

17. A process of preparing a pharmaceutical composition for oral contraception and simultaneous blood pressure regulation and for reducing the risk of folate deficiency-induced congenital malformations in the event of pregnancy, said process comprising including
a combination of 2.0 mg of dienogest, 0.030 mg of ethinyl estradiol, and (6S)-5-methyltetrahydrofolate; or
a combination of 2.0 mg of dienogest, 0.020 mg of ethinyl estradiol, and (6S)-5-methyltetrahydrofolate; or
a combination of 1.5 mg of dienogest, 0.015 mg of ethinyl estradiol, and (6S)-5-methyltetrahydrofolate; in the pharmaceutical composition.

18. The process as defined in claim 17, wherein 451 μg of a crystalline or a micro-encapsulated calcium salt of (6S)-5-methyltetrahydrofolic acid is included in the pharmaceutical composition.
